(19) United States
(12) Patent Application Publication
(10) Pub. No.: US 2026/0249039 A1
(43) Pub. Date: Aug. 27, 2026

(54) Wearable ASMR Device

(71) Applicant: Thellisra Wellness LLC, St. Charles, IL (US)

(72) Inventors: Deanna Jean Chrones, St. Charles, IL (US); Michael Anthony Meuer, Elburn, IL (US); James Charles Bartelsen, Nashotah, WI (US)

(21) Appl. No.: 19/552,239

(22) Filed: Feb. 27, 2026

Related U.S. Application Data
(60) Provisional application No. 63/763,983, filed on Feb. 27, 2025.

(51) Int. Cl. A61M 21/02; A61M 21/00

(52) U.S. Cl. CPC A61M 21/02; A61M 2021/0022; A61M 2021/0066; A61M 2205/0233; A61M 2205/07; A61M 2205/106; A61M 2205/13; A61M 2205/3673; A61M 2210/10

(57) ABSTRACT

A wearable ASMR device helps to stimulate release certain neurochemicals in the human body by eliciting autonomous sensory meridian response (“ASMR”) to treat mental health issues such as depression, anxiety, trauma, and other mental disorders, and to create relaxation and feelings of general wellness. The device may stimulate ASMR in numerous ways, as different people may attain ASMR through different physical actions. A neck device has an adjustable sliding element that moves across the back of the neck. A shoulder or back panel device has electroconductive strips to contract and relax targeted muscles in an adjustable cycle such as ramping up an electrical charge to a maximum level over a first period of time, holding the electrical charge at the maximum level for a second period of time, ramping down the electrical charge for a third period of time, then pausing the electrical charge for a fourth period of time.

### CROSS-REFERENCE TO RELATED APPLICATIONS

This application claims priority to U.S. Provisional Application No. 63/763,983 filed Feb. 27, 2025, and incorporated herein by reference in its entirety.

### FIELD OF THE INVENTION

The present invention relates to a wearable therapeutic/medical device.

### BACKGROUND OF THE INVENTION AND PRIOR ART

ASMR may trigger the release of neurochemicals that result in feelings of relaxation, peace, and safety, as well as additional therapeutic effects. Thus, the target users are people who suffer from mental disorders (e.g., depression, anxiety, trauma, etc.) and receive counseling or psychotherapy, as well as people in the general population who wish to benefit from improving their stress, relaxation, sleep, focus, and overall state of health.

Currently, ASMR is only achieved by seeking sound and/or visual triggers. Typically, this means going to websites to, for example, listen to soft sounds (such as whispering, or a feather brushing over a microphone) or watch a video by a wellness professional. These existing methods of attaining ASMR are not easily doable, nor is ASMR even attainable by many people through these methods; neither are these methods convenient for incorporation into psychotherapy sessions.

No other known devices exist to physically achieve ASMR by cyclically contracting the middle and upper trapezius muscles around the spinal column; cyclically contracting the upper trapezius muscles along the spine; and/or mimicking the feeling of gentle touch, cold metal, the graze of hair on the skin, and/or a cold breeze around the neck. Nor are there any ASMR devices of any kind known to be on the market. The present invention creates a reliable and convenient means of achieving ASMR. An additional advantage of the present invention is that it may facilitate psychotherapeutic treatment of patients who may have experienced sexual or physical abuse, without requiring them to remove their clothing or be touched by another person.

### BRIEF DESCRIPTION OF THE FIGURES

FIG. 1 is a front perspective view of a neck device.

FIG. 2 is a front perspective, cross-sectional view of a neck device.

FIG. 3 is a front perspective view of a slider and piston mechanism.

FIG. 4 is a partial view of a slider and filament brush.

FIG. 5 is a front perspective view of two panels with handles and cables.

FIG. 6 is a rear view of the two panels with handles and cables shown in FIG. 5.

FIG. 7 is an exemplary procedure for an adjustable cycle of muscle stimulation via panels.

FIG. 8 is a block diagram of a method of operating the neck device.

FIG. 9 is a block diagram of a method of operating the neck device in conjunction with the panels.

FIG. 10 is an exemplary layout for a user application.

### DESCRIPTION OF THE INVENTION

The present disclosure is directed to a wearable device that may help stimulate release certain neurochemicals in the human body in order to treat mental health issues such as depression, anxiety, trauma, and other mental disorders, and to create relaxation and feelings of general wellness. The present disclosure is meant to elicit autonomous sensory meridian response (“ASMR”), the tingling sensation felt in the brain and/or the body, usually upon receiving gentle touch or personal or social grooming and attention, the known effects of which include feelings of relaxation, peace, and safety, even when physical sensations do not occur. The neurochemicals that are released as a result of ASMR are also known to produce additional therapeutic effects. The device may stimulate ASMR in numerous ways, as different people attain ASMR through different physical actions. The present disclosure may also be used to treat other medical diseases and/or their symptoms.

The present disclosure contemplates two main groups of actions to achieve ASMR. One group of actions is performed by the Neck Device; the other group of actions are performed separately by the Shoulder Panels or the Back Panels, which are attachments that may plug into the Neck Device and may be operable separate from the Neck Device. In one embodiment, the Neck Device is used only for controlling and/or positioning the Shoulder Panels or Back Panels, and the Neck Device need not be otherwise operational. Both groups of actions that create ASMR are varied and numerous, because each person achieves ASMR in their own way. This allows individuals to customize the actions, selecting the action(s) that work best for them, or to experience any or all of the actions in randomized fashion. In other words, in one embodiment, the Wearable ASMR Device has a single, main component (the “Neck Device”), and in another embodiment, a different component (the “Back Panels”) can be attached to the Neck Device. In yet another embodiment, another different component (the “Shoulder Panels”) can be attached to the Neck Device, including in conjunction with the Back Panels.

The Neck Device, Back Panels, and Shoulder Panels are described herein. The Neck Device simulates actions known to cause ASMR in humans by, including, for example, mimicking gentle touch, the feel of cold metal, the graze of hair(s) on the skin, and/or a cold breeze. These actions can be conducted in random order, as randomization and unexpectedness also mimic the human experience and enhances ASMR. The Back Panels and Shoulder Panels stimulate the contractions of certain, targeted muscles. The Back Panels, for example, move the middle and upper trapezius muscles so said muscles gently “hug,” or slightly wrap around, the spinal column, which may elicit ASMR therapeutically. The Shoulder Panels, for example, stimulate the upper trapezius or “shrug” muscles, to move said muscles up and down along the spine, which may also elicit ASMR therapeutically.

All actions of the Neck Device, Back Panels, and Shoulder Panels may be controlled by a software app with on-screen images, spoken guidance, and monitoring for ease of use.

The first group of actions simulates actions known to bring on ASMR, such as, for example, receiving personal grooming or attention from another person, a gentle touch to the skin or styling of the hair, and the feel of cold metal or a cold breeze against the skin. However, touch by another person is most often, or completely, contraindicated in mental health therapy. Therefore, the Neck Device replicates, for example, the sensation of a gentle touch that moves across the skin, the feel of cold metal against the skin, the graze of a single hair or a strand of hair against bare skin, and a cold breeze, without human touch. The Neck Device can also replicate said exemplary sensations in random order, as the uncertainty and unexpectedness of randomization itself also replicates the human experience and increases ASMR. The Neck Device is further described herein.

The other group of actions is the contraction and movement of certain muscles. Specifically, the muscles along the top half of the spinal column are activated to contract and then relax, and therefore, to create movement along or around the spinal column. This creates a “massaging” effect on the spinal column/spine that produces ASMR.

The Neck Device replicates the physical sensations described above, for example, as follows:

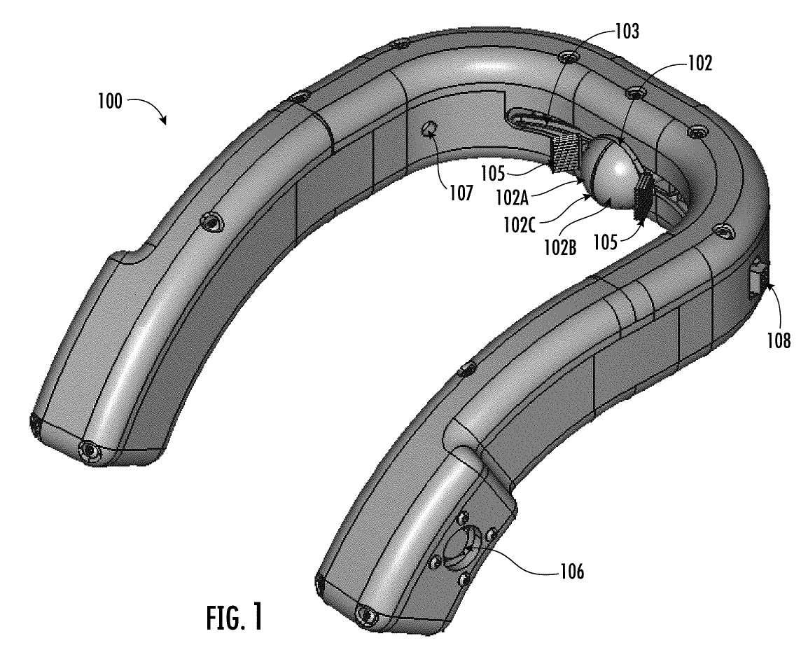

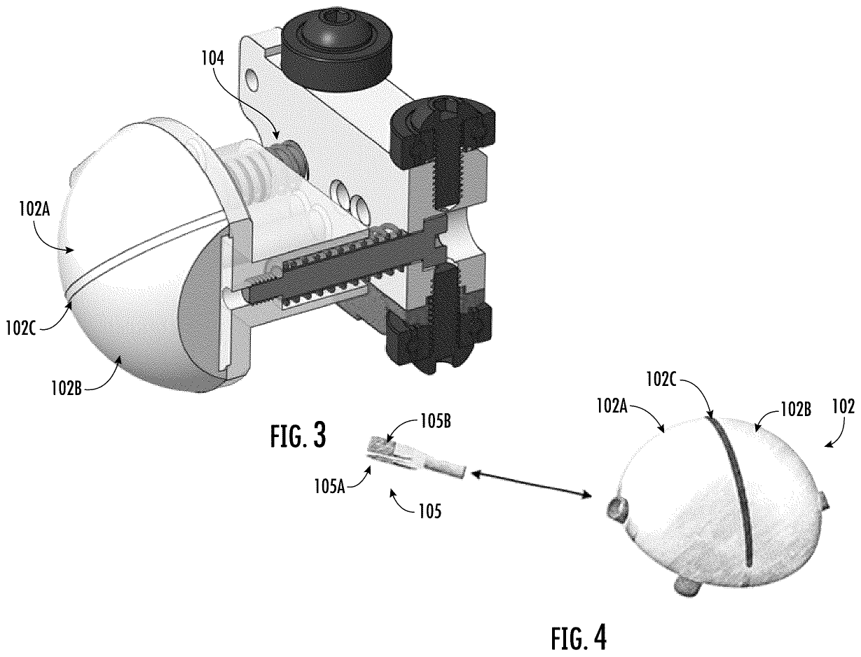

The Neck Device comprises a casing or housing (100) that is shaped to rest around the neck, with its two ends that may rest on a user's shoulders in front of the collar bones, as seen in FIG. 1. The Neck Device further comprises an electrically chilled or warmed, rounded piece of metal (“Metal Slider”) (102) that moves along a track (103), such as a rail, belt, or slot in the housing, against the skin on the back of the neck in both directions, for varying lengths of distance, at varying speeds, in random fashion to simulate random, human-like, soothing actions, and to simulate a cool or warm touch and the feeling of smooth, metal, and to stimulate ASMR. The Metal Slider (102) may be on a piston mechanism (104), servo, or other articulating means, such as shown in FIG. 3, which moves the Metal Slider (102) toward and away from the skin randomly, to enhance that randomized, “unexpected” effect and deepen ASMR. In one embodiment of the invention, the “chilling” or piezoelectric cooling of the Metal Slider (102) is created by a small Peltier module or something similar. There may be cooling gel or a Peltier circuit inside the sliding metal to cool the Metal Slider to below body/room temperature. The Metal Slider (102) may also be electrically warmed.

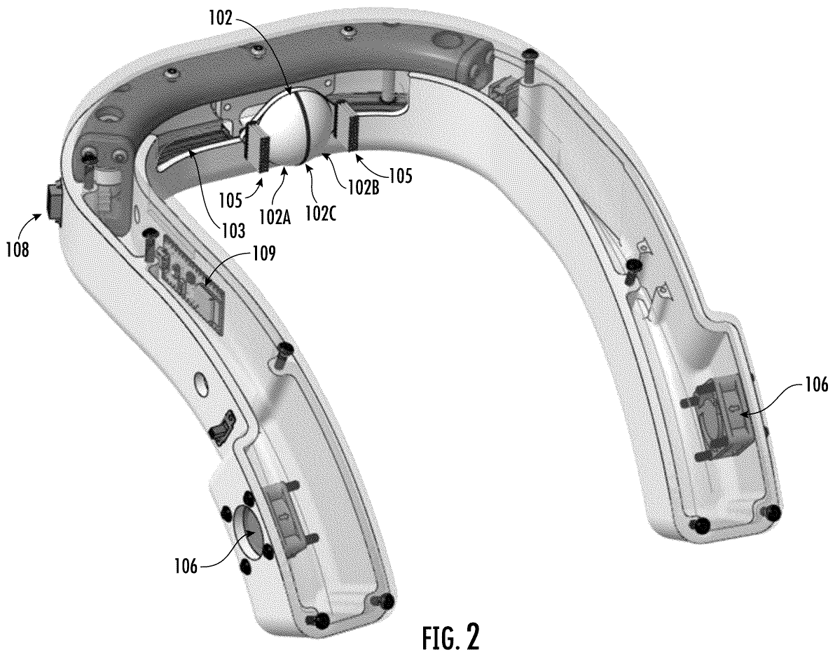

The present invention also comprises one or more processors (109) and a memory, storing executable instructions, which when executed by the processor(s) (109) control operation of the neck device and/or panels (200) including determining skin contact (for example via impedance logic or infrared sensors) and operating the Metal Slider (102), fans (106), brushes (105), and/or estim panels (200) and strips (210). Thus, the processor(s), such as shown in FIG. 2, can also assess whether the Metal Slider (102) is connecting with the skin and is maintaining a desirable pressure on the skin (neither too hard and dragging nor pressing too lightly and not touching). This is achieved by “dividing” the rounded Metal Slider into two pieces (102A, 102B), with an insulator (102C) between the two halves. This allows for measuring the impedance between the two halves. Different impedance measurements indicate varying levels of connection and pressure. For example, if the impedance measurement is infinite, it is not touching the skin.

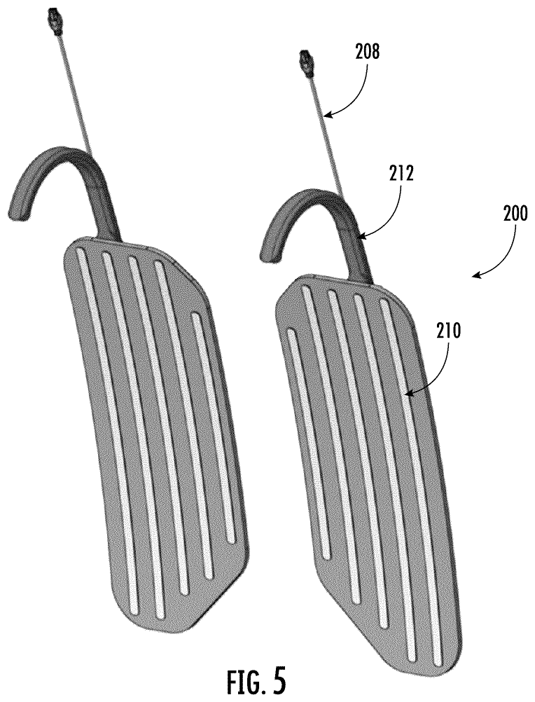

Additionally, on each side of the Metal Slider (102), there may be a structure that resembles the tips of two or more small makeup brushes of different sizes that are connected to each other side-by-side, for a total of four or more “makeup brushes” (105) as shown in FIGS. 1, 3, and 4. These two double-brush structures (105) on either side of the Metal Slider (102) are referred herein as “Brushes.” These Brushes (105) can include soft, strong filaments that are gentle enough to be hair-like, to mimic the sensation of small strands of hair just grazing the skin. This combined brush-and-ball structure (102, 105) moves back and forth along the track (103), touching the skin—and forward and back with the piston (104) allowing them to move toward and away from the skin—in a simulated random fashion. This is done to increase the likelihood of achieving ASMR, and the natural variability of movements of such hair-like filaments (105) further heightens the randomness. Alternatively, a user can select a continuous (i.e., without randomness) motion option for the Neck Device or other adjustable lengths and speeds.

Filament Brushes (105) have the appearance of bundled “makeup brushes.” Within this bundle, one of the two “makeup brushes” is a thin brush (105A), with just a few “hairs” or filaments (for example, a very small brush that traces a thin line, approximately 1 mm wide at the tip, or less, with the longest filaments in the center, making it a very delicate, tipped brush). Bundled next to that thin brush (105A), but with empty space between the two sets of bristles (e.g., 2-4 mm), may be a larger brush (105B) (for example, with bristles that are cut straight across) that traces a wider, but still narrow line (approx. 8-9 mm wide), over the skin. The larger brush (105B) is for those with less skin sensitivity, who might not feel the smaller brush. In certain embodiments, the larger brush (105B) may have its bristles or filaments cut straight across, perpendicular to the “line” it traces across the skin. The bristles can be made of various materials, including, for example, synthetic materials, and can include anti-microbial properties. The combined structure of the Metal Slider (102) and Filament Brushes (105) (the “Moveable Structure”) comprises removeable, cleanable, and replaceable components for hygiene and preference.

The Metal Slider (102) or the Movable Structure (102, 105) may move back and forth along the track against the skin on the back of the neck, for varying distances, at varying speeds, and in random fashion. For example, the speed may be between 1 cm per second and 5 cm per second. Additionally, for example, the distance may vary between 1 cm and 40 cm. The piston or servo (104) may also have a variable rate of movement.

The Neck Device may also have fans (106) at each end, which simulate a cool breeze on the skin to further enhance ASMR and keep the device cool in this version of the invention.

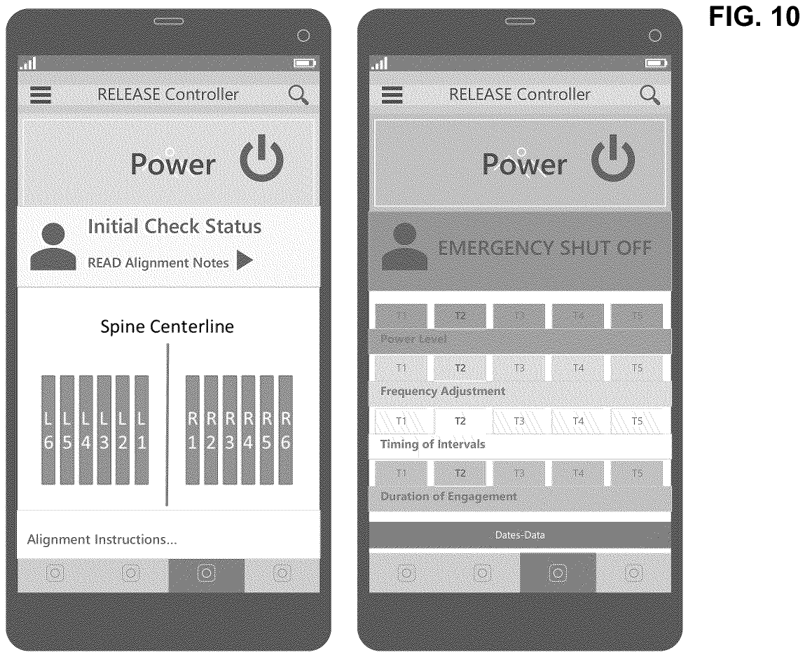

The Neck Device can host the power source for the overall device (i.e., the Neck Device or Neck Device in conjunction with Shoulder Panels and/or Back Panels (200)), which can take the form of a rechargeable battery or wired connection. The Neck Device also holds the controller circuitry, to control actions performed by the Metal Slider (102), Moveable Structure (102, 105), the Shoulder and/or Back Panels (200), along with communicating with a user application/user input panel to further guide/control such defined actions, as shown in FIG. 10.

The software/user application can also allow the user to choose from a selection of sounds already known to produce ASMR, and/or that the spoken guidance will have a “meditative” effect (e.g., performed in a whisper), to enhance the ASMR produced by the device for some users. It is important to note that impedance values may vary significantly from each other in consecutive flow. An initial test run of the Moveable Structure (102, 105) back-and-forth across the entire neck may indicate the presence of large amounts of body hair on the neck or skin imperfections that might otherwise interfere with the successful use of the device. Therefore, in another embodiment of the invention, the precise amount or level of such increased variability in readings in such situations, or the act of encountering one or more significant skin imperfections, would need to be determined. But once determined, the programming informs the piston (104) not to move all the way to the skin, but rather to stay further away, with the intention of grazing the hairs on the skin, and of then enhancing piloerection of hairs on skin, for even further ASMR generation, or with the intention of grazing the skin imperfection(s) more gently than might occur by full skin contact, also for enhanced ASMR, or not at all if problematic. This can also be accomplished without the Brushes (105) attached, using just the Metal Slider (102), which is smooth, to prevent entanglement and enhance ASMR, relying on the piloerection effect on body hair in cold temperatures, intensified by the skimming of the hairs produced by the sensors/impedance-LIM-V measurements. The cooling by the Peltier module, device fans (106), or by some other means can produce the cold temperatures also needed for this enhanced effect.

Because of the importance of accurate readings of the skin topography, another embodiment of the present invention includes the use of infrared sensors (107) to measure impedance, instead of or in addition to the impedance-determining structure of the Metal Slider (102) described earlier (i.e., with split-globe slider design, whose halves (102A, 102B) are adhered by insulating material (102C)). Such infrared sensors also allow for determining the Moveable Structure's relative distance from the skin, body hair, or skin features and for attaining proper contact, or lack thereof, continuously, including and making any necessary adjustments to this distance to maximize ASMR for the user. The addition of such infrared sensors (107) allows for the initial test run of the Neck Device (i.e., the first time back-and-forth across the neck) to be done with a fully retracted piston (104), which can prevent hair entanglement or skin-feature irritation. In addition to the split-globe design of the Metal Slider (102) and the infrared sensors (107), other measures of impedance and distance-from-skin can be used.

The other main group of actions (i.e., the muscle contractions/relaxations) are created by the two Shoulder Panels and/or the two Back Panels, in other words, the panels (200). The panels are separably operable in conjunction with the Neck Device.

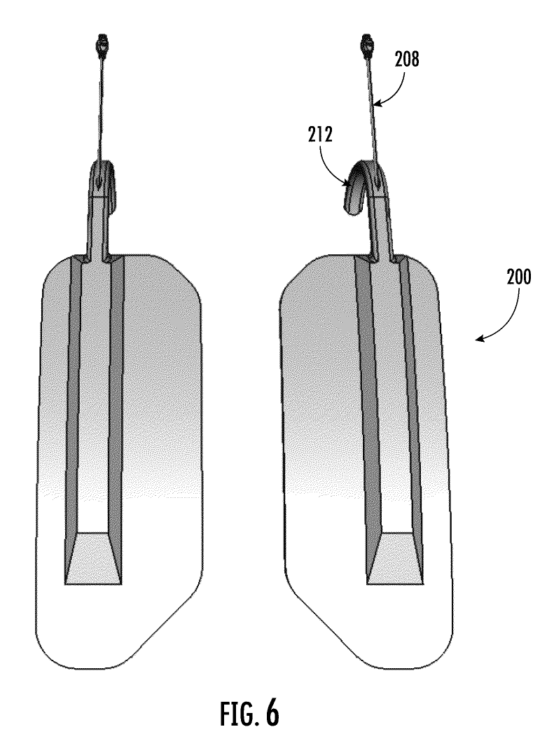

The Shoulder Panels and Back Panels (200), shown in FIGS. 5 and 6, use electrical muscle stimulators, herein referred to as E-Stim.

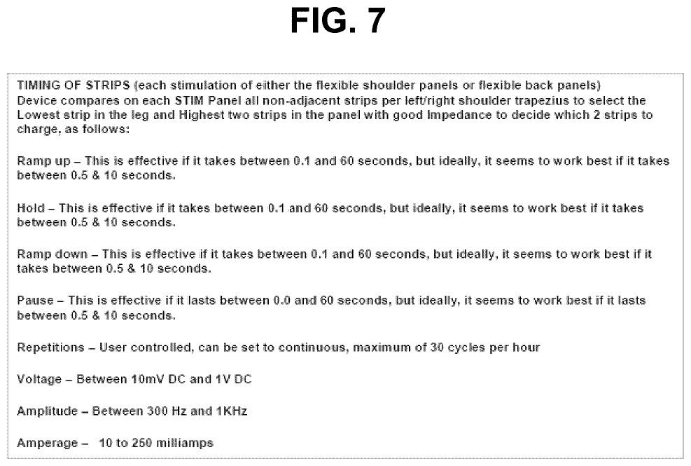

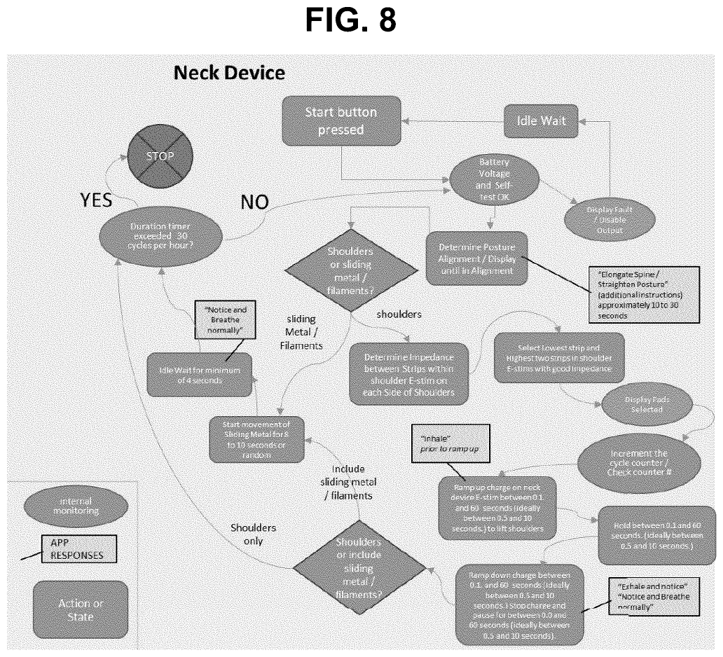

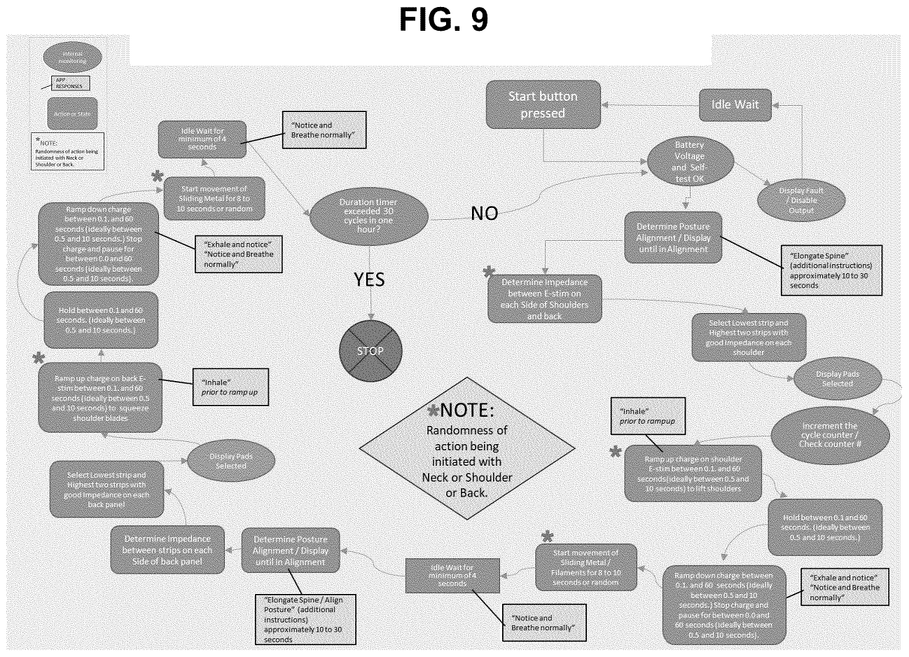

The Panels use E-Stim to stimulate muscles (for example, the upper trapezius / “shrug”, lifting the shoulders up and down along the spine) in an adjustable cycle (ex. the timing shown in FIG. 7). The adjustable cycle comprises ramping up an electrical charge to a maximum level over a first period of time, holding the electrical charge at the maximum level for a second period of time, ramping down the electrical charge for a third period of time, then pausing the electrical charge for a fourth period of time.

The Shoulder Panels consist of at least two panels (200), including at least one for placement on each side of the user's neck, and each individual Shoulder Panel (200) may connect to the Neck Device via one or more connector ports (108) and cables (208). Each Shoulder Panel may be placed on either side of the Neck Device in various positions. These variable placement options assure proper placement on the muscle, wherein proper movement and successful placement of the Shoulder Panels (200) is shown and guided by the software and user input. Each Shoulder Panel (200) holds snug to the shoulders through the tight fit achieved when it is connected into the Neck Device, or otherwise moved into the proper place. In another embodiment, the Shoulder Panels (200) are integrated with the Neck Device, rather than being separate components.

The Shoulder Panels (200) have one or more features ensuring firm contact with the skin that is necessary for stimulating the muscle contractions. For example, the E-Stim strips (210) may be covered by a flexible-but-firm plastic or plastic-like material, use a spring or a hinge, and/or are attached via a belt or other feature that wraps, for example, under the armpit and back up to the panel (200).

Each Panel (200) has a set of strips (210), wherein each strip is made of an electro-conductive material, such as silver cloth, for example, and each strip (210) is electrically isolated from each other, as shown in FIG. 5. Each Shoulder Panel (200) also includes circuitry for measuring impedance and stimulating muscle contractions via the strips. The strips (210) on the Shoulder Panels (200) both monitor and send charges to retract/adduct the targeted muscles (for example, the upper trapezius, which is mid-way between base of neck and the clavicle bone on the top of the shoulder) or just below the user's shoulders on the back, to raise the user's shoulders for a determined period. The panels (200) apply a low-voltage frequency to one strip (210) and detect the frequency in the other strips (210) to determine an impedance value. This impedance value is used to determine the strength of contact between each E-stim strip (210) and the user. The identification of these contacts can be used to select the appropriate muscles to stimulate. The E-Stim strips (210) are then used to stimulate the selected muscles. The level and timing of stimulation/charge is adjustable to move the muscles sufficiently to achieve desired sensation, while maintaining safe use. This is meant to mimic the physical exercises which can be executed without the device in many people (but could be enhanced in those people) and achieved in others (who otherwise cannot achieve it) by using the device.

In one embodiment, there are at least four E-Stim strips (210) per Shoulder Panel (200) and the size of each E-Stim strip (210) should accommodate at least one square inch of contact per strip.

In another embodiment, a user application/user input panel, as shown, for example, in FIG. 10, can be used to control and monitor the Shoulder Panels, and collect data.

The Back Panels'E-Stim (i.e., sensors/stimulators) (210) sends charges to retract/adduct the muscle known as the middle and upper trapezius (as opposed to the tops of the upper trapezius muscles only, as in the Shoulder Panels) to squeeze/pinch the shoulder blades (scapulae) towards each other. The level of stimulation/charge is adjustable to move the muscles sufficiently to achieve the desired sensation, while maintaining safe use. This mimics physical exercises which can be executed without the device in many people (but could be enhanced in those people) and achieved in others (who otherwise cannot achieve it) by using the device.

The Panels (200) may be made of a flexible plastic or a flexible plastic-like material that is not electro-conductive, such as polycarbonate, and can be held tightly against the shoulder blades and back region of the body. The electro-conductive strips (210) that are attached to it are likewise composed of a conductive, yet flexible material, such as painted/printed carbon, copper tape, or silver cloth, for example.

In another embodiment, a user application or user input panel, as shown, for example, in FIG. 10, can be used to control and monitor the Back Panels, and collect data.

In one embodiment of the present invention, sensors that sense proper posture for the back of neck/spine and sends signal/notification regarding proper alignment can be used to enhance the actions of both the Back Panels and the Shoulder Panels. These sensors can be placed in the Neck Device (for example, approx. between C2-C6), because elongating the spinal column enhances these physical ASMR exercises/muscle movements.

The Panels can be slid under the clothing, without removal of clothing. For example, the Shoulder Panels would likely only need to be slid partway under clothing, likely without any special accommodations, though a handle (212) or some other mechanism might be used, to guide and place the panels. To achieve placement of the Back Panels (200) over the shoulder blades, the panels may have a long, flexible handle (212) that extends from one top corner of each panel, for a length that may be for example 6 inches to 14 inches, as shown in FIGS. 5 and 6. The panels'Handles (212) might also be adjustable in length, being made shorter or longer during placement/use, as needed. The Back Panel for the user's left side might have a handle extending from the top, possibly located all the way at the left top corner and the Back Panel for the user's right side might have a handle extending from the top, possibly located all the way to the right top corner. Each Handle (212) will help the User guide the panel (200)—under the clothing—and move it around, so it can cover the proper location over the shoulder blades, one panel at a time; then the Handle (212) can be manually curved down over the top of the shoulder, to hold the panel (200) in place.

After that, the User may apply—over the clothes—a wrap, or a series of straps, or a vest, or some combination of these, or some other shape, to secure the Panels (200) in place in their location under the clothes and to keep them from moving. This will allow for psychotherapeutic treatment of a patient/User, within the therapy room, who might have experienced sexual or physical abuse, without necessitating the User to remove their clothing or needing much assistance to place the device, as such undressing or touching would be contraindicated in this and many other, if not all, cases. To assist with sliding the Back Panels (200) under clothing and undergarments, the panels may have a smooth, tapered edge at the bottom, opposite the Handle (212), that will be thin enough and smooth enough to slide under clothes. Likely, the four corners of each panel may be rounded in this embodiment. One or more of the Panel (200) components may be disposable.

In one embodiment, the overall device (whether just the Neck Device, or the Neck Device with Back/Shoulder Panels) is limited to performing a maximum of 30 cycles of movement or device-generated opportunities to attain ASMR per hour. For example, if a the user is not using any of the panels (200), the device would then “count” only the actions of the Moveable Structure (102, 105) or Metal Slider (102) of the Neck Device that result in direct skin contact or in the pre-determined grazing of the hairs or imperfections on the skin, toward the maximum of 30 cycles per hour, again resulting in a maximum of 30 possible device-driven opportunities to attain ASMR per hour. The maximum of 30 cycles applies to one cycle of the E-Stim Panel set (Back or Shoulder) (200), or one movement of the Moveable Structure (102, 105) or Metal Slider (102) with the piston (104) in some level of forward position (either directly touching the skin for some random length/rate of speed or by grazing any hairs or skin imperfections, with the Piston (104) at a less “advanced” position, for some random length/rate of speed, described above). In other embodiments, the maximum number of cycles could be set at higher or lower than 30 cycles per hour.

The present invention may be used for clinical testing of ASMR, touch, coldness, and the related neural and neurochemical states, on mice and other animals, for research on medical illnesses such as various forms of dementia, and Parkinson's disease, among other medical disorders. The previously described embodiment with enhanced impedance and distance measures (e.g., for addressing large amounts of body hair on the neck), would also allow for the use of the device with fur, for animal testing, using the device in miniaturized form, for example, with testing mice who have been programmed with Alzheimer's disease to be treated with the soothing “touch” of the device. The controls in such an embodiment would be Bluetooth and remote and would be managed by the researcher(s). Beyond just miniaturization of the device, one such embodiment might also include use of the Metal Slider (102) alone, rather than with Filament Brushes (105), and it might be placed over the back of the animal, rather than the neck. The previously described options for measuring impedance and distance from skin would be of especially great importance here, as mice and other animals show great flexibility of muscle movement; grazing—and of only some parts of the fur, given the animal's natural movements—might be the only option for use. However, it is possible that fuller contact with the rodent's fur and skin, by the rounded Metal Slider (102) (without the Filament Brushes), may also work.

What is claimed is:

1. A wearable device comprising:
a curved housing comprising a track;
a slider having a rounded shape oriented away from the curved housing and configured to move along the track;
one or more processors;
a memory storing executable instructions that, when executed by the one or more processors, control operation of the slider based on user input.

2. The wearable device of claim 1, wherein the slider is metal.

3. The wearable device of claim 1, wherein the slider is electronically chilled or heated.

4. The wearable device of claim 1, wherein the slider is configured to move at adjustable lengths and at adjustable speeds along the track, including in a random fashion.

5. The wearable device of claim 1, wherein the slider is configured to move in a direction perpendicular to the track.

6. The wearable device of claim 1, wherein the track comprises a slot defined in the curved housing.

7. The wearable device of claim 1, wherein the slider further comprises infrared sensors, wherein the instructions, when executed by the one or more processors, determine impedance based on signals received from the infrared sensors.

8. The wearable device of claim 1, wherein the slider comprises two halves with an insulator between the two halves.

9. The wearable device of claim 1, further comprising at least one filament bundle adjacent to the slider and configured to move with the slider.

10. The wearable device of claim 1, wherein at least one end of the curved housing has a fan.

11. A wearable device comprising:
at least two panels;
wherein each of the at least two panels comprises at least two strips;
wherein the at least two strips comprise an electroconductive material;
wherein each of the at least two strips are electrically isolated from each other;
wherein the at least two panels are configured to electrically stimulate muscle contraction during an adjustable cycle; and
wherein the adjustable cycle comprises ramping up an electrical charge to a maximum level over a first period of time, holding the electrical charge at the maximum level for a second period of time, ramping down the electrical charge for a third period of time, then pausing the electrical charge for a fourth period of time.

12. The wearable device of claim 11, wherein the at least two panels comprise a flexible, non-electroconductive material.

13. The wearable device of claim 11, wherein at least one panel further comprises at least one handle.

14. The wearable device of claim 11, wherein the first period of time, the second period of time, the third period of time, and the fourth period of time are each between 0.5 and 10 seconds.

15. A wearable device comprising:
a curved housing comprising a track;
a slider having a rounded shape oriented away from the curved housing and configured to move along the track; and
at least two panels;
wherein each of the two panels comprises at least two strips;
wherein the at least two strips comprise an electroconductive material;
wherein each of the two panels is configured to electrically stimulate muscle contraction.

16. The wearable device of claim 15, wherein the slider is metal.

17. The wearable device of claim 16, wherein the slider is electronically chilled or heated.

18. The wearable device of claim 15, wherein the slider is configured to move for varying lengths and at varying speeds along the track in a random fashion.

19. The wearable device of claim 15, wherein the at least two panels comprise a flexible, non-electroconductive material.

20. The wearable device of claim 15, wherein the at least two panels are configured to electrically stimulate muscle contraction in an adjustable cycle, wherein the adjustable cycle comprises ramping up an electrical charge to a maximum level over a first period of time, holding the electrical charge at the maximum level for a second period of time, ramping down the electrical charge for a third period of time, then pausing the electrical charge for a fourth period of time.
